(19) United States
(12) Patent Application Publication
(10) Pub. No.: US 2015/0090674 A1
(43) Pub. Date: Apr. 2, 2015

(54) BLOOD FILTER APPARATUS FOR SEPARATING PLASMA OR SERUM FROM BLOOD AND USE OF THE BLOOD FILTER APPARATUS

(71) Applicant: Samsung Electronics Co., Ltd., Suwon-si (KR)

(72) Inventors: Tae-han LEE, Yongin-si (KR); Sang-hyun Baek, Yongin-si (KR); Hyo-young Jeong, Yongin-si (KR); Young-Ki Hahn, Seoul (KR); Soo-suk Lee, Suwon-si (KR); Youn-suk Choi, Yongin-si (KR)

(21) Appl. No.: 14/301,014

(22) Filed: Jun. 10, 2014

(30) Foreign Application Priority Data
Oct. 1, 2013 (KR) 10-2013-0117591

(51) Int. Cl. A61M 1/34

(52) U.S. Cl. CPC A61M 1/3496; A61M 2205/75; A61M 2205/7554
USPC 210/797; 210/335; 210/806

(57) ABSTRACT

A blood filter apparatus for separating plasma or serum from blood, a cartridge for analyzing blood including the blood filter apparatus, and a method of separating plasma or serum using the blood filter apparatus.

### CROSS-REFERENCE TO RELATED APPLICATIONS

This application claims the benefit of Korean Patent Application No. 10-2013-0117591, filed on Oct. 1, 2013, in the Korean Intellectual Property Office, the entire disclosure of which is hereby incorporated by reference.

### BACKGROUND

1. Field

The present disclosure relates to a blood filter apparatus for separating plasma or serum from blood, cartridges for analyzing blood using the blood filter apparatus, and methods of separating plasma or serum from blood using the blood filter apparatus.

2. Description of the Related Art

A variety of separation membranes have been proposed for removing corpuscle components from blood to obtain plasma or serum required for clinical tests. In general, plasma or serum may be separated, but the separation rate thereof was slow. When pressure was applied to blood to improve the separation rate, hemocatheresis, hemolysis, or erythrocyte diapedesis may result, and, accordingly, such damaged corpuscles may also contaminate the separated plasma or serum. In addition, in blood in which fibrin precipitates, clogging of the blood is more likely to occur during the separation, and thus hemolysis may be easily caused in the blood.

Blood separation filters are classified according to horizontal separation methods and vertical separation methods. A blood separation filter using a horizontal separation method separates whole blood which flows slowly along a filter by gravity or capillary forces. A blood separation filter using a vertical separation method separates whole blood in a way that the whole blood is dropped into the filter, and then pressurized by applying pressure from the flow direction of the whole blood or from the reverse flow direction of the whole blood. In general, there have been many studies on horizontal separation methods. When the whole blood is separated by the blood separation filter using a vertical separation method, there are advantages of separating the whole blood in a short time in accordance with a short distance for the whole blood to flow along the filter. However, such methods may result in problems with filter clogging and hemolysis, and thus the combination of filters and the pressure conditions are considered important.

In general, erythrocytes occupy about 45% of the whole blood and about 98% of solid components. Thus, when 1% of erythrocytes are subject to hemolysis, changes occur not only in electrolytes, including 24.4% potassium and 1.0% sodium, but also in concentrations of clinical factors such as LDH, GOD, GPT, glucose, and inorganic phosphate. Thus, a low occurrence of hemolysis is important in regard to chemical analysis of the blood.

Therefore, there is a demand for miniaturization of blood diagnostic apparatuses and a method of separating blood using a filter for a blood test.

### SUMMARY

Provided is a blood filter apparatus for separating plasma or serum from a blood sample, the blood filter apparatus comprising a plurality of filter members and a plasma or serum separation membrane, wherein the plurality of filter members are serially connected to each other and disposed on the plasma or serum separation membrane, and each of the plurality of filter members have a filled density of about 0.49 to about 0.65 g/cm³.

Also provided is a cartridge for analyzing blood comprising the blood filter apparatus, the cartridge comprising a testing unit configured to receive a blood sample and a housing including at least one supply hole configured to supply the blood sample to the testing unit, wherein the blood filter apparatus is disposed in the supply hole of the housing.

Further provided is a method of separating plasma or serum from a blood sample using the blood filter apparatus, the method comprising providing a blood sample to the blood filter apparatus and compressing the filter members of the blood filter apparatus.

### BRIEF DESCRIPTION OF THE DRAWINGS

These and/or other aspects will become apparent and more readily appreciated from the following description of the embodiments taken in conjunction with the accompanying drawings, in which:

FIG. 1 is a front view schematically illustrating a blood filter apparatus;

FIG. 2 is a graph showing the degree of hemolysis and recovery rate during blood injection according to filled density and diameter of a filter member of the blood filter apparatus;

FIG. 3 is a graph showing the degree of hemolysis and recovery rate depending on pressurization time and pressure with respect to types of the filter member in the blood filter apparatus;

FIGS. 4 and 5 are graphs showing measurable hematocrit (HCT) ranges with respect to compositions of the filter member in the blood filter apparatus;

FIG. 6 is a schematic view illustrating a cartridge for analyzing blood;

FIGS. 7A, 7B, and 7C are plan views each illustrating a cartridge for analyzing blood, which includes at least one through hole;

FIG. 8 is a side-sectional view schematically illustrating a cartridge for analyzing blood;

FIG. 9A is an exploded perspective view illustrating each layer of a testing unit in the cartridge for analyzing blood, FIG. 9B is a plan view schematically illustrating a upper plate of the testing unit in the cartridge for analyzing blood, and FIG. 9C is a plan view schematically illustrating a lower plate of the testing unit in the cartridge for analyzing blood;

FIGS. 10A to 10E are plan views each schematically illustrating a middle plate of the testing unit in the cartridge for analyzing blood;

FIGS. 11A to 11D are plan views each schematically illustrating a middle plate that may include a microfluidic structure; and

FIG. 12 is a plan view illustrating a middle plate including two inlets.

### DETAILED DESCRIPTION

Reference will now be made in detail to embodiments, examples of which are illustrated in the accompanying drawings. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an aspect of the present invention, a blood filter apparatus includes a plurality of filter members, and a plasma or serum separation membrane, wherein the plurality of the filter members may be serially connected to top of the plasma or serum separation membrane. The plasma or serum separation membrane may be a membrane to separate plasma or serum from blood.

The term “serially connected” used herein is not limited to the case where subjects are directly connected, and thus the term may refer to the case where any other members are disposed between the subjects.

Each of the plurality of the filter members may be sequentially stacked and bonded to each other. The filter member may be layer that is designed to physically block certain objects or substances while letting others through, and be made of a porous material such as a porous polymer material. The filter member may be made from woven fibers or non-woven fibers. The non-woven fibers may include a homogenous blend or mix of fibrillated and non-fibrillated synthetic staple fibers. Also, the filter member may include binder material used in the fabrication of the fiber. One or more (or all) of the plurality of the filter members may be a glass fiber filter (e.g., comprising a glass fiber filter material). The glass fiber filters may have a surface coated with a polymer. Alternatively, the glass fiber filters may have a surface coated with a polymer after being washed out with acid.

The blood filter apparatus may be used to obtain plasma or serum with a high recovery rate from a blood sample such as whole blood. Also, the blood filter apparatus may be used to reduce the occurrence of hemolysis of corpuscles including erythrocytes. The blood filter apparatus may include a filter member in a predetermined filled density that may obtain plasma or serum with a high recovery rate. The term “filled density” may mean the mass of many particles of a filter member per unit volume, and the unit volume may include the particle volume, inter-particle volume, and internal pore volume (see e.g, U.S. Pat. No. 7,744,820 B, U.S. Pat. No. 7,927,810 B, U.S. Pat. No. 7,993,847 B). Also, the filled density may be referred to as a “bulk density”. The filter member may have a filled density in a range from about 0.49 to about 0.65 g/cm³, for example, about 0.49 to about 0.61 g/cm³, about 0.49 to about 0.56 g/cm³, about 0.53 to about 0.65 g/cm³, or about 0.53 to about 0.56 g/cm³.

The filter member may have a weight per surface area (or a basis weight) in a range from about 40 to about 150 g/m², for example, about 50 to about 140 g/m², about 60 to about 130 g/m², about 70 to about 120 g/m², about 80 to about 110 g/m², or about 90 to about 100 g/m². The filter member may have a thickness in a range from about 0.8 to about 1.2 mm, for example, or about 0.9 to about 1.1 mm. For example, the filter member may have a thickness of about 1.0 mm. The filter member may have a diameter in a range from about 7 to about 7.7 mm, for example, about 7.1 to about 7.6 mm, about 7.2 to about 7.5 mm, or about 7.3 to about 7.4 mm.

The filter member may have a particle retention size or a pore size in a range from about 1.25 to about 2.75 μm, for example, about 1.5 to about 2.5 μm, or about 1.75 to about 2.25 μm. For example, the filter member may have a particle retention size or a pore size of about 2.0 μm.

The filter member may have a property of adsorbing fibrinogen. The filter member may be formed of glass fiber. In addition, a material forming the fibrinogen-adsorbing filter member may be polyester-based resin such as polyethyleneterephthalate and polybutyleneterephthalate; nylon resin; polyurethane resin; polystyrene-based resin; resins composed of homopolymer or copolymer of polymethacrylic acid ester; or resin composed of copolymer of polyethylene and vinyl acetate or methacrylic acid ester.

The plasma or serum separation membrane may be a membrane for separating plasma or serum from blood. The plasma or serum separation membrane may have a plurality of through holes penetrating from one side of the membrane to the other. A planar shape of an opening of the through hole and a cross section shape of the through hole may be a curved shape such as circle or ellipse. A longitudinal section of the through hole along the extending direction of the through holes may include an inside wall in a linear or curved shape. In addition, the extending direction of the through holes may be orthogonal to the surface of the membrane, or may be inclined from the orthogonal direction. The longitudinal section of the through hole may be in a cut, truncated cone shape. A method of forming the through hole may include energy beam irradiation such as ion beam irradiation, or chemical treatments such as alkaline erosion, after the membrane formation is completed.

The through hole may have a diameter in a range from about 0.2 to about 1.0 μm, for example, about 0.3 to about 0.9 μm, about 0.4 to about 0.9 μm, about 0.5 to about 0.9 μm, about 0.6 to about 0.9 μm, or about 0.7 to about 0.9 μm. For example, the through hole may have a diameter of about 0.8 μm. When the through hole has a diameter less than 0.2 μm, proteins or lipids in the blood are likely to clog the through hole. When the through hole has a diameter greater than 1.0 μm, corpuscles such as erythrocytes may pass through the membrane due to their deformability.

In addition, the plasma or serum separation membrane to separate plasma or serum from the blood may have a porosity in a range from about 10 to about 20%, for example, in a range from about 11 to about 19%, about 12 to about 18%, about 13 to about 17%, or about 14 to about 16%. For example, the plasma or serum separation membrane may have a porosity or about 15%.

The plasma or serum separation membrane may be a microporous membrane, and the microporous membrane may include an isotropic membrane, an anisotropic membrane, an asymmetric membrane, a membrane including both asymmetric and isometric regions, and/or a composite membrane. An isometric membrane may have a porous structure in which pores whose size is substantially the same with an average pore size are distributed through an interior of the membrane. For example, in regard to the average pore size, an isomeric membrane may have a structure in which pores whose size is substantially the same with the average pore size are distributed throughout the entire membrane. An asymmetric membrane may have a pore structure, which varies throughout the inner membrane. For example, the average pore size may be reduced to a size of a portion or another portion of a surface or to a size of the surface. For example, the average pore size may be reduced to a size of an upstream side or a downstream side of the surface or to a size of the surface. The microporous membrane may include a track etched membrane (TEM), a fibrous mesh membrane, or a cast membrane.

The microporous membrane may react or bond to a specific substance, or may include a functional membrane on which a functional material that absorbs a specific substance is coated. The functional material may be a compound including at least one functional group selected from a functional group containing carbon and hydrogen, such as alkane, alkene, alkyne, or arene; a halogen atom-containing functional group such as a halogen compound; a hydrogen-containing functional group such as alcohol or ether; a nitrogen-containing functional group such as amine or nitrile; a sulfur-containing functional group such as thiol or sulfide; and a carbonyl group-containing functional group such as carbonyl, aldehyde, ketone, carboxylic acid, ester, amide, carboxylic acid chloride, or carboxylic acid anhydride.

The microporous membrane may include a plurality of pores, and may further include at least one of porous membranes filtering a substance whose size is greater than the above-described pore and contained in the blood sample. The mesoporous membrane may include a polymer membrane selected from polycarbonate (PC), polyethersulfone (PES), polyethylene (PE), and polysulphone (PS), and polyarylsulfone (PASF). The filter member may have a structure in which the functional material that reacts with a specific material in the fluid sample is filled between at least one of the microporous membranes. The microporous membrane may have a porosity in a range selected from about 1:1 to about 1:200.

The microporous membrane may include at least one polyester and polycarbonate. The plasma or serum separation membrane may be formed of materials selected from any one of synthetic polymers and natural polymers. Examples of the materials include cellulose mixed ester, polyvinylidene difluoride, polytetrafluoroethylen, polycarbonate, polypropylene, polyester, nylon, glass, or alumina.

The track etched membrane (TEM) may be formed of a plastic membrane, and the plastic may be formed of a polymeric material. The track etching may involve bombarding a solid film with particles to form weakened tracks (see e.g., U.S. Pat. No. 6,103,119 A). Examples of the polymeric material include polyester, polystyrene, aromatic polyester, polycarbonate, polyolefine, vinyl plastic such as polyvinyl difluoride (PVDF), or cellulose ester. Examples of the polyolefine include polyethylene, polyethylene terephthalate, or polypropylene. The pores within the TEM may be formed by a process of etching selectively performed on a damaged film using gas or liquid, and the pore size may be determined by a residence time of the etchant.

The blood filter apparatus may further include a compressing device, and the compressing device may be disposed on the filter member of the blood filter apparatus. The compressing device, which is a means for compressing the filter member, may be any structure that can apply pressure to the filter members to compress the filter members against the separation membrane, for example, a plunger.

The blood sample may be a whole blood sample or a diluted blood sample. The blood may be from human blood or animal blood. In addition, the blood may be fresh blood or blood mixed with anticoagulates such as heparin, ethylenediamine tetraacetate, or citric acid.

According to another aspect of the present invention, provided is a cartridge for analyzing blood to perform examination on a blood sample. The cartridge for analyzing blood may include a testing unit where a blood sample is introduced and tested, a housing including at least one supply hole supplying the blood sample to the testing unit, and the above-described blood filter apparatus that is disposed on the supply hole of the housing.

The housing may further include a gripping portion formed in a streamlined shape on the opposite side of the supply holes. The housing may be formed of materials selected from the group consisting of polymethylmethacylate (PMMA), polydimethylsiloxane (PDMS), polycarbonate (PC), linear low density polyethylene (LLDPE), low density polyethylene (LDPE), medium density polyethylene (MDPE), high density polyethylene (HDPE), polyvinyl alcohol, very low density polyethylene (VLDPE), polypropylene (PP), acrylonitrile butadiene styrene (ABS), cycloolefin copolymer (COO), glass, talc, silica and semiconductor wafer. The bottom surface of one side of the housing may be bonded to the top surface of one side of the testing unit.

The testing unit may include an inlet in which the fluid sample is introduced from the supply holes, and the housing and the testing unit may be bonded to correspond to the supply holes and the inlet, respectively. The testing unit may include a plurality of testing chambers for testing the fluid sample that is introduced from the inlet; and a supply flow channel connecting the inlet and the plurality of the testing chambers. The supply flow channel may have a width in a range from about 1 to about 500 μm. The testing unit may include an upper plate, a lower plate, and a middle plate that is disposed between the upper plate and the lower plate, wherein the plates are formed in a film shape. The upper plate and the lower plate may be formed of at least one film selected from the group consisting of polyethylene films such as very low density polyethylene (VLDPE), linear low density polyethylene (LLDPE), low density polyethylene (LDPE), medium density polyethylene (MDPE), and high density polyethylene (HDPE), polypropylene (PP) films, polyvinyl chloride (PVC) films, polyvinyl alcohol (PVA) films, polystyrene (PS) films, and polyethylene terephthalate (PET) films. The middle plate may be formed of a porous sheet. The upper plate, the middle plate, and the lower plate may each have a thickness in a range from about 10 to about 300 μm. The inlet, the plurality of the testing chambers, and the supply flow channel may be formed on the middle plate. The upper plate and the lower plate may be printed with light-shielding ink, and the upper plate and the lower plate may include regions corresponding to the plurality of the testing chambers, and the regions may be treated to be transparent.

The housing may include at least two supply holes, and the testing unit may include at least two inlets in a position corresponding to at least two of the supply holes.

According to another aspect of the present invention, provided is a method of separating plasma or serum from the blood sample, the method including providing the blood sample to the above-described blood filter apparatus; and applying pressure to compressing device of the above-described blood filter apparatus.

The applied pressure may be in a range from about 7 to about 9 kPa, for example, about 7.3 to about 8.7 kPa, or about 7.5 to about 8.5 kPa. The applying of the pressure may be performed for about 10 to about 16 seconds, for example, about 12 to about 15 seconds, or about 13 to about 15 seconds. The blood sample may be whole blood having hematocrit (HCT) less than 55%. The blood sample may be whole blood having a volume in a range from about 70 to about 100 μl.

In some embodiments, when hemolysis occurs in a concentration of about 10 mg/dL, 40% or more of the plasma or serum may be separated. In addition, the plasma or serum may be separated within 25 seconds, for example, in 15 to about 25 seconds.

The method may further include a process of washing the blood filter apparatus with water or acid. In the blood filter apparatus, the blood filter apparatus may be washed out when an amount of the residual electrolyte in the blood filter apparatus reaches a predetermined amount. The acid may be acetic acid, hydrochloric acid, or sulfuric acid.

Hereinafter, the present invention will be described in further detail with reference to the following examples. These examples are for illustrative purposes only and are not intended to limit the scope of the invention.

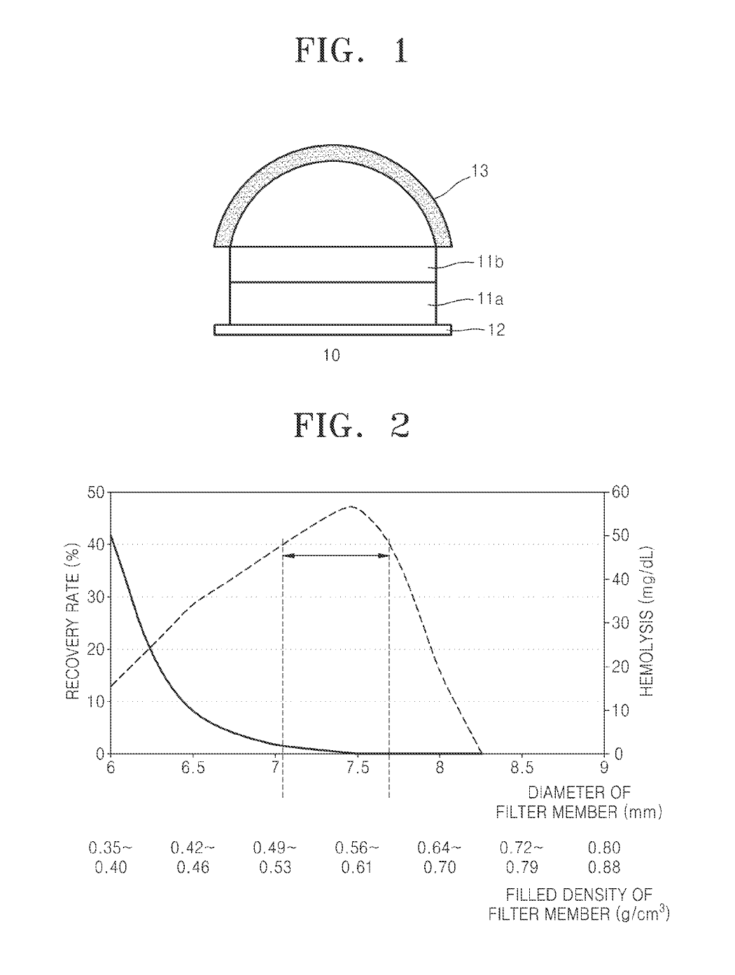

FIG. 1 is a front view schematically illustrating a blood filter apparatus. As shown in FIG. 1, a blood filter apparatus 10 includes a plurality of filter members 11a and 11b and a plasma or serum separation membrane 12 for separating plasma or serum from blood.

The plurality of the filter members 11 may include, for example, a first filter member 11a and a second filter member 11b, wherein a top surface of the first filter member 11a may be serially connected with a bottom surface of the second filter member 11b. The first filter member 11a and the second filter member 11b may be serially connected on top of the plasma or serum separation membrane 12. For example, the first filter member 11a and the second filter member 11b may be sequentially stacked on top of the plasma or serum separation membrane 12, with one filter member (e.g., 11a in FIG. 1) connected with membrane 12. Here, the surface of the first filter member 11a may have the same diameter with the second filter member 11b. The first filter member 11a and the second filter member 11b may have the same or different thickness.

Each of the plurality of the filter members 11 may have a filled density in a range from about 0.49 to about 0.65 g/cm³. When the plurality of the filter members 11 has a filled density less than about 0.49 g/cm³ or greater than about 0.65 g/cm³, recovery rate of the separated plasma or serum may be decreased. In addition, when the plurality of the filter members 11 has a filled density less than about 0.49 g/cm³, the load applied on erythrocytes may become large, and accordingly hemolysis is more likely to occur.

The plurality of the filter members 11 may have a weight by surface area (specific surface area) in a range from about 40 to about 150 g/m². Also, the plurality of the filter members 11 may have a total thickness equal to or greater than a depth of the supply holes in cartridge for analyzing blood. The plurality of the filter members 11 may have a total thickness equal to the depth of the supply holes, or plurality of the filter members 11 may have a total thickness less than about 1 mm. For example, the plurality of the filter members 11 may have a total thickness in a range from about 0.8 to about 1.2 mm. When the plurality of the filter members 11 has a total thickness less than the depth of the supply holes, the plasma or serum separation membrane may become loose and absorb the pressure transferred from the compressing device, and at the same time, may trap the separated blood within the plasma or serum separation membrane 12. The plurality of the filter members 11 may have a diameter in a range from about 7 to about 7.7 mm. Each of the plurality of the filter members 11 may include a cross section in a circular or elliptical shape. Each of the plurality of the filter members 11 may include may include a particle retention size in a range from about 1.25 to about 2.75 μm.

The plasma or serum separation membrane 12 may include a plurality of the through holes penetrating from one side of the membrane to the other. More specifically, the plasma or serum separation membrane 12 may include the plurality of the through holes, wherein each of the plurality of the through holes may have a diameter in a range from about 0.2 to about 1.0 μm. The plasma or serum separation membrane 12 may have a porosity in a range from about 10 to about 20%

The blood filter apparatus may further include a compressing device 13 on top of the plurality of filter members 11. The compressing device 13 may compress the blood sample disposed on top of the plurality of filter members 11. The compressing device 13 may be, for example, a plunger.

FIG. 6 is a schematic view illustrating a cartridge for analyzing blood. As shown in FIG. 6, a cartridge for analyzing blood 100 may include a housing 110 and a testing unit 120.

The housing 110 may support the cartridge for analyzing blood 100, and include a gripping portion 112 for a user to grip the cartridge for analyzing blood 100. The gripping portion 112 may be formed in a shape to facilitate gripping. For example, the gripping portion 112 may be formed in a streamlined, protruded shape.

The housing 110 may include a blood supply unit 111 for receiving the blood sample. The blood supply unit 111 may include a supply hole 111a through which the supplied blood sample flows into the testing unit 120. The supply hole 111a may have a circular or polygonal shape. In addition, the blood supply unit 111 may further include an auxiliary supply unit 111b assisting the supply of the blood sample. The auxiliary supply unit 111b may be formed around the supply hole 111a so as to be inclined to the direction of the supply hole 111a, to assist in flowing the blood sample into the supply hole 111a.

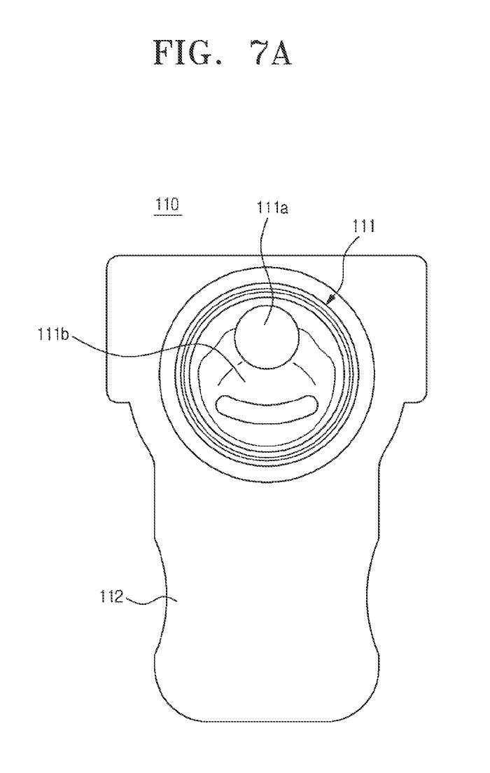

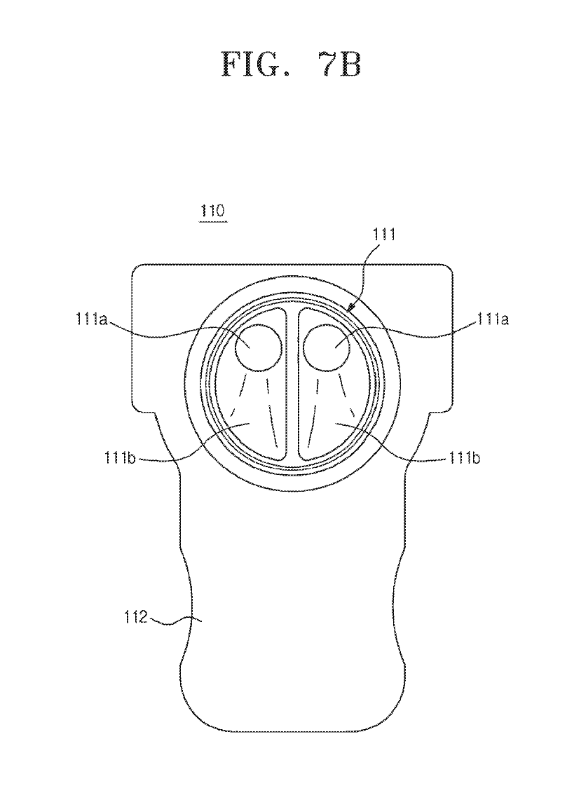

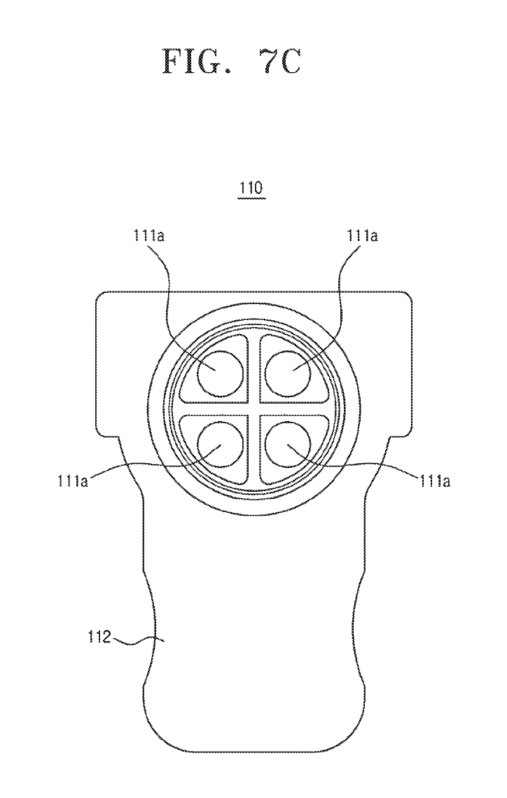

FIGS. 7A, 7B, and 7C are plan views each illustrating the housing of the cartridge for analyzing blood 100 (FIG. 6), which includes at least one through hole. As shown in FIGS. 7A and 7B, the housing 110 may include at least one supply hole 111a and at least one supply auxiliary unit 111b. For example, as shown in FIG. 7B, the housing 110 may include one, two, or four supply holes 111a and one, two, or four supply auxiliary units 111b, respectively. The supply hole 111a may have a diameter in range from about 0.5 to about 10 mm, for example, about 0.5 to about 8 mm, about 0.5 to about 6 mm, about 0.5 to about 4 mm, about 0.5 to about 2 nm, or about 0.5 to about 1 mm.

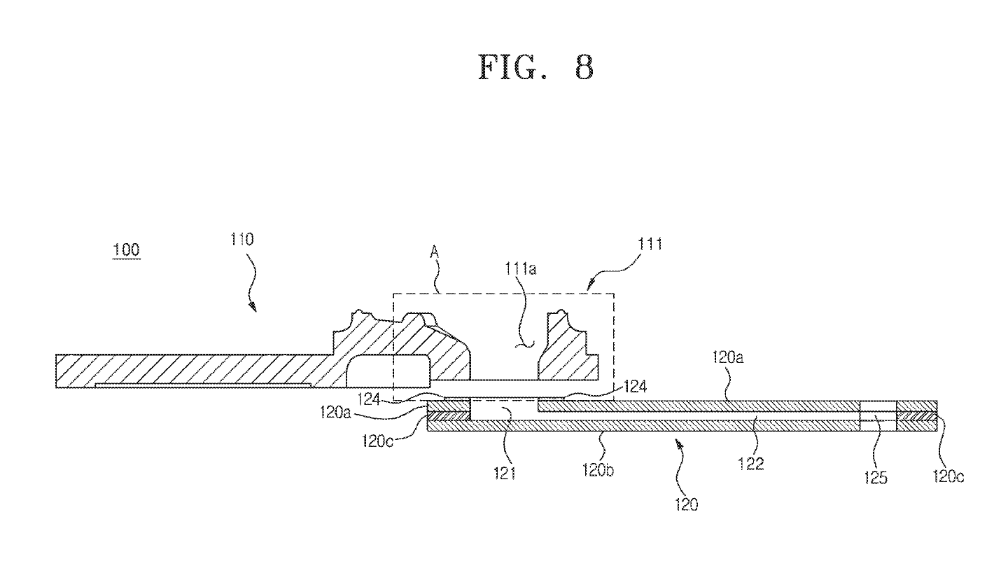

FIG. 8 is a side-sectional view schematically illustrating the cartridge for analyzing blood 100. As shown in FIG. 8, the cartridge for analyzing blood 100 may be formed in such a manner that the blood supply unit 111 is attached to the testing unit 120 on the bottom part of the housing 110. The housing 110 may be bonded to the testing unit 120 by a pressure sensitive adhesive (PSA) or a double-sided adhesive. Alternatively, the housing 110 may be bonded to the testing unit 120 in a way that a protrusion part is fitted into a groove.

As shown in FIG. 8, region A is a region where the blood filter apparatus 10 of FIG. 1 may be coupled to the cartridge for analyzing blood 100 through the supply hole 111a. The blood filter apparatus 10 may be fitted inside of the supply hole 111a.

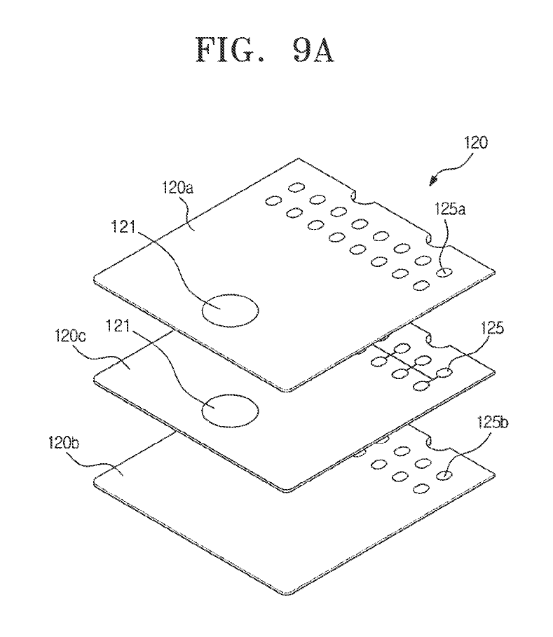

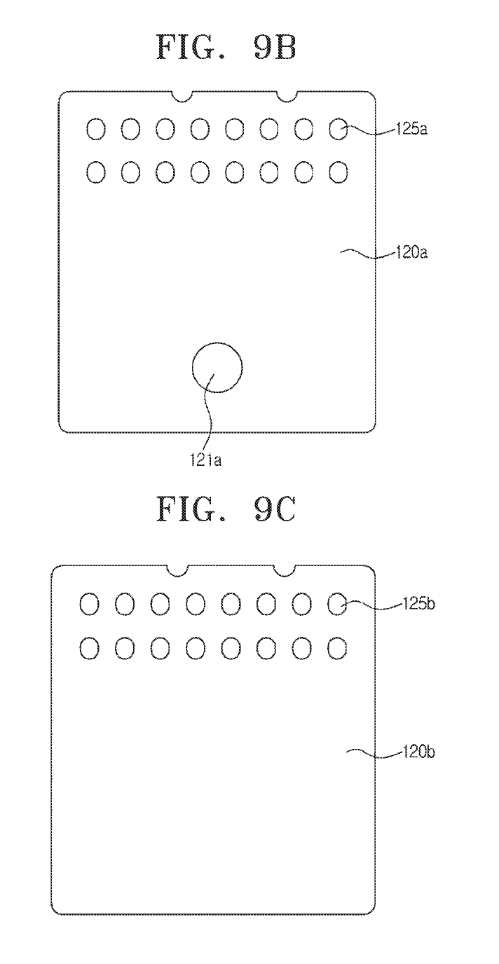

FIG. 9A is an exploded perspective view illustrating each layer of the testing unit 120 in the cartridge for analyzing blood 100. FIG. 9B is a plan view schematically illustrating an upper plate 120a of the testing unit 120, and FIG. 9C is a plan view schematically illustrating a lower plate 120b of the testing unit 120. As shown in FIG. 9A, the testing unit 120 of the cartridge for analyzing blood 100 may be formed in a structure consisting of an upper plate 120a, a lower plate 120b, and a middle plate 120c that are bound to each other. The upper plate 120a and the lower plate 120b may be printed with light-shielding ink, and thus the fluid sample flowing into a testing chamber 125 may be protected from external light or errors that may be caused when measuring optical properties in the testing chamber 125. The upper plate 120a and the lower plate 120b may be formed in a film shape. The film may include a material that is chemically and biologically inert and has mechanical processability. The film may be selected from the group consisting of polyethylene films, PP films, PVC films, PVA films, PS films, and PET films. The polyethylene films may further include VLDPE films, LLDPE films, LDPE films, MDPE films, or HDPE films.

The testing unit 120 may include the middle plate 120c as a porous sheet such as cellulose, which is different from materials of the upper plate 120a and the lower plate 120b. The middle plate 120c may function as a vent. Also, the middle plate 120c may promote movement of the blood sample within the testing unit 120 without requiring an additional driving source.

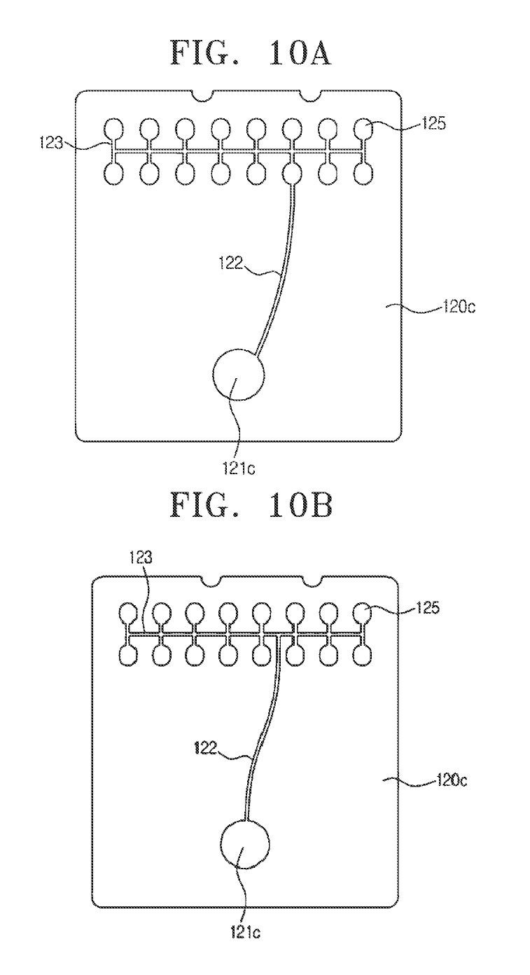

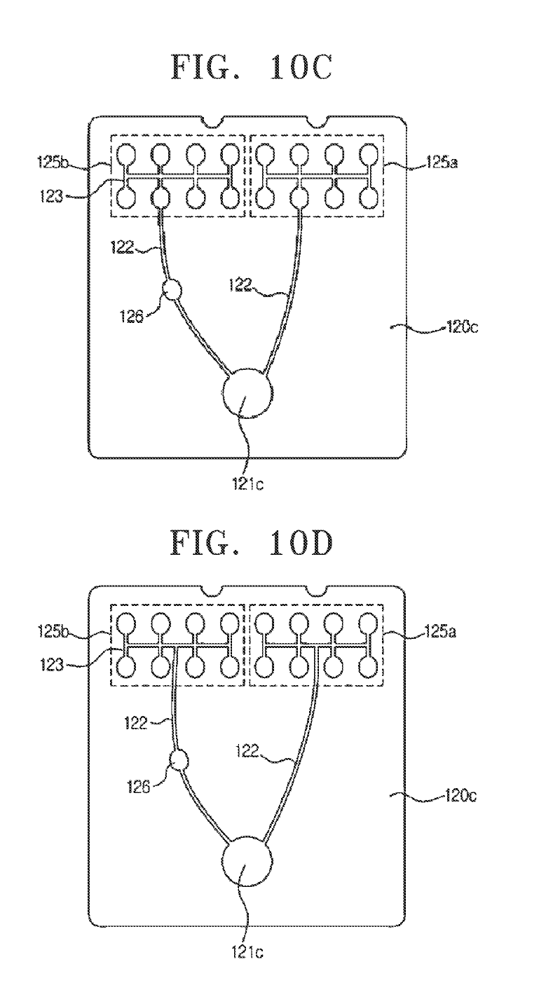

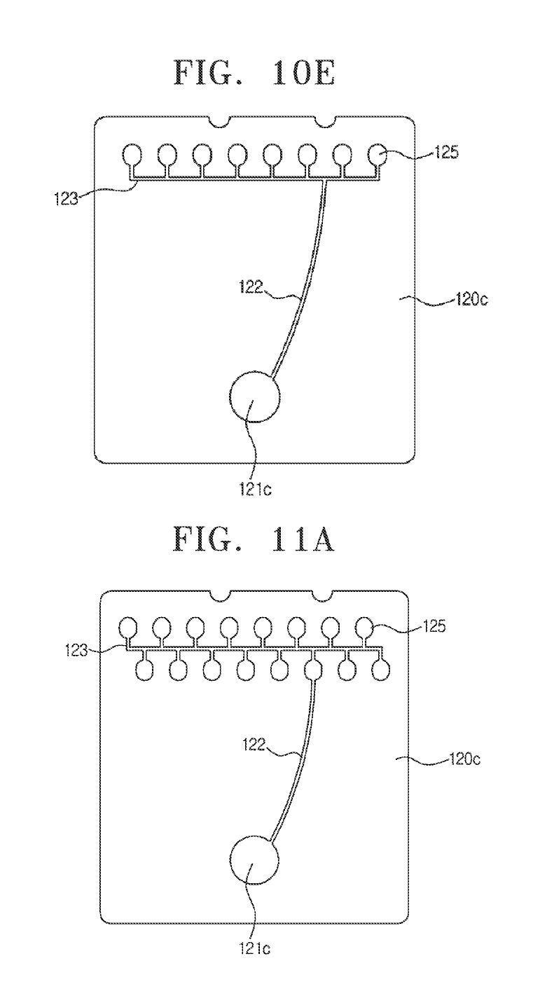

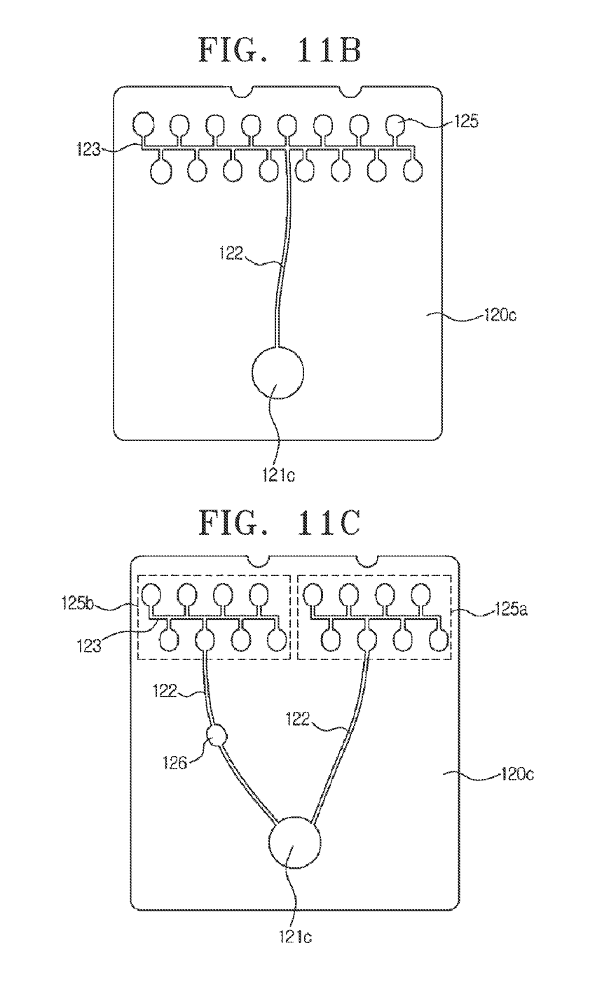

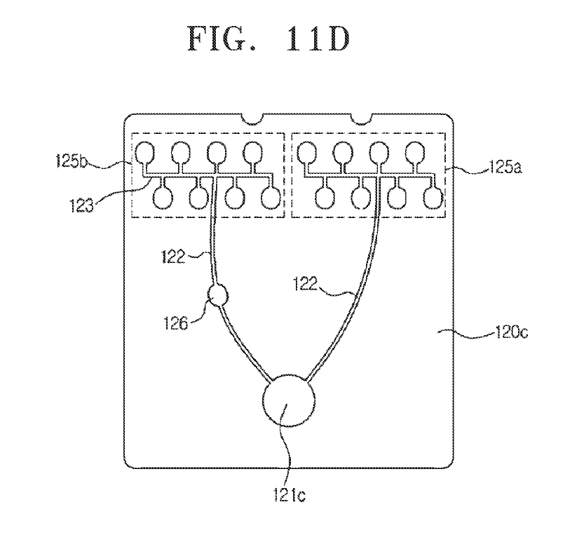

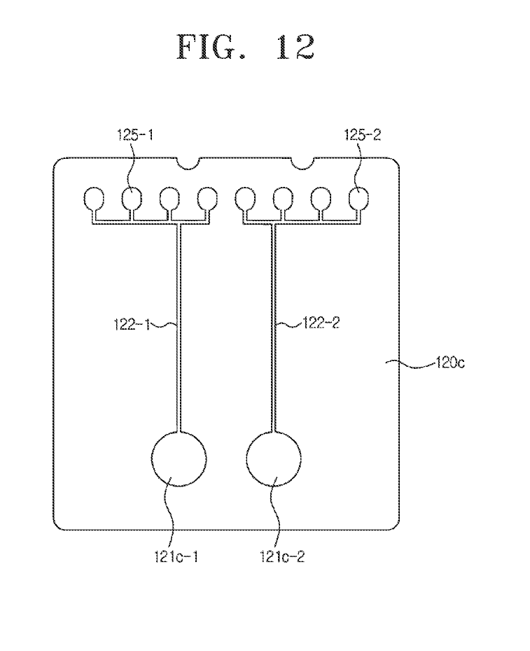

Referring again to FIG. 8, the microfluidic structure that may be formed on the testing unit 120 may include an inlet 121 through which the blood sample is introduced via the blood filter apparatus 10 (FIG. 1), a supply flow channel 122 (shown in FIGS. 10A-11D) transferring the introduced blood sample to the testing chamber 125, and the testing chamber 125 where a reaction between the blood sample and a reagent occurs. Such microfluidic structure of the testing unit 120 may be formed on the middle plate 120c.

Referring to FIGS. 9A to 9C, when the testing unit 120 has a three-layer structure, the upper plate 120a may include an inlet 121a through which the blood sample is introduced, and a region 125a corresponding to the testing chamber 125 may be treated to be transparent. In addition, the lower plate 120b may include another region 125b corresponding to the testing chamber 125, which may be treated to be transparent. These regions 125a and 125 b that correspond to the testing chamber 125 may be used to measure optical properties of the reaction occurring in the testing chamber 125

As shown in FIG. 10A, the middle plate 120c may include an inlet 121c through which the blood sample is introduced. When the upper plate 120a, the middle plate 120c, and the lower plate 120b are bonded to each other, the inlet 121a of the upper plate 120a and the inlet 121c of the middle plate 120c may overlap so as to form the inlet 121 (FIG. 8) of the testing unit 120.

The middle plate 120c may include the testing chamber 125 in a region on the opposite side of the inlet 121c. For example, in the middle plate 120c, a region corresponding to the testing chamber 125 may be removed in the shape of a circle or square so as to form the testing chamber 125. Since regions 125 of the upper plate 120a and the lower plate 120b each corresponding to the testing chamber 125 are not removed, a certain region may be removed in the middle plate 120c so as to form the testing chamber 125 to accommodate the blood sample and the reagent. The removed region in the middle plate 120c may form a hole therein, and the hole may form the testing chamber 125. In addition, a fine storage container may be disposed in the removed region in the middle plate 120c to be used as the testing chamber 125.

In this regard, a variety of reactions for analyzing blood may occur in the testing chamber 125. For example, the testing chamber 125 may accommodate the reagent in advance, which may be colored or discolored as it reacts with a specific component of the separated plasma or separated serum. Then, colors that are expressed in the testing chamber 125 may be optically detected and quantified. Accordingly, the presence or absence of the specific component in the blood sample or a ratio between the specific component and the blood sample may be measured. The middle plate 120c may include the supply flow channel 122 supplying the blood sample introduced from the inlet 121c to the testing chamber 125. The supply flow channel 122 may be formed when a region corresponding to the supply flow channel 122 is removed in the middle plate 120c. The supply flow channel 122 may be formed to have a width in a range from about 1 to about 500 μm.

As shown in 10A, the supply flow channel 122 may connect the inlet 121c with one of the plurality of the testing chambers 125. The blood sample introduced from the inlet 121c may pass through the supply flow channel 122 by a capillary force to reach one of the plurality of the testing chambers 125. Then, the blood sample may pass through a branch channel 123 again by a capillary force to reach each one of the plurality of testing chambers 125, wherein the branch channel 123 connects each of the plurality of the testing chambers 125, and thus the blood sample may react with reagent that was accommodated in advance in the testing chambers 125. The plurality of the testing chambers 125 that are directly connected to the inlet 121c by the supply flow channel 122 may be in an empty state or may accommodate the reagent or the reaction solution to perform pretreatment on the fluidic sample.

As shown in FIG. 10B, in some embodiments, the supply flow channel 122 may be connected to the branch channel 123 instead of one of the plurality of testing chambers 125. That is, according to types of the blood sample or examination performed in the testing chamber 125, the supply flow channel 122 may be connected with one of the plurality of the testing chambers 125 or with the branch channel 123. When the upper plate 120a of FIG. 9B, the lower plate 120b of FIG. 9C, and the middle plate 120c of FIG. 10A are bonded with each other, one complete testing unit 120 may be formed. Accordingly, the testing unit 120 and the housing 110 may be bonded to each other, thereby forming the cartridge for analyzing blood 100.

Also, as shown in FIGS. 10C and 10D, in some embodiments the inlet 121c may be connected with two separate supply flow channels 122. In this case, the plurality of the testing chambers 125 may be separated into two separate testing regions 125a and 125b. When a middle chamber 126 is formed on any one of the two supply flow channels 122, pretreatment may be performed in one testing region 125b which is connected by the supply flow channel 122 having the middle chamber 126. Alternatively, in one testing region 125b, the fluidic sample which has previously undergone the first reaction may be supplied. In some other embodiments, the middle chamber 126 may be formed on each of the two separate supply flow channels 122, and accordingly two different pretreatments may be performed in each middle chamber 126, or the first reaction with different reagents and reaction solutions may occur in each middle chamber 126. In some other embodiments, three or more supply flow channels 122 may be connected to one inlet 121c, and accordingly the blood sample is supplied to three or more testing regions.

As shown in FIG. 10E, the plurality of the testing chambers 125 may be arranged in a single layer structure. In this case, the transparent regions from the upper plate 120a and the lower plate 120b may be formed in a position corresponding to the plurality of the testing chambers 125.

As shown in FIG. 11A, the plurality of the testing chambers 125 may be arranged top and bottom so as to form a bilayer structure, wherein the plurality of the testing chambers 125 on the top and the plurality of the testing chambers 125 on the bottom may intersect each other like a zigzag. In this case, the blood sample may be supplied to the time difference. When the blood sample passes through one of the plurality of the testing chambers 125, and is then distributed to the rest of the plurality of the testing chambers 125, the reagent or the reaction solution for pretreatment of the blood sample may be accommodated in the testing chamber 125 through which the blood sample is first passed. In some embodiments, the testing chamber 125 directly connected with the supply flow channel 122 may be in an empty state.

As shown in FIG. 11B, the supply flow channel 122 connected with the inlet 121c may not be directly connected to one of a plurality of testing chambers 125. Instead, the supply flow channel 122 may be connected with the branch channel 123. As described above, it may be determined, with respect to types of the blood sample or examination performed in each of the plurality of the testing chambers 125, whether the supply flow channel 122 connected with the inlet 121c is connected with one of the plurality of the testing chambers 125 or with the branch channel 123 to then be distributed in the rest of the plurality of testing chambers 125.

As shown in FIGS. 11C and 11D, the plurality of the testing chambers 125 may be divided into two separate testing regions 125a and 125b. Two separate supply flow channels 122 may be connected to the corresponding testing regions 125a and 125b. Each of the two supply flow channels 122 may connect the inlet 121c with each of the two separate testing regions 125a and 125b. One of the two supply flow channels 122 may include the middle chamber 126 between the inlet 121c and the testing region 125b, and thus the fluidic sample may pass through the middle chamber 126 so as to perform pretreatment or the first reaction in the middle chamber 126. Alternatively, two separate middle chambers 126 may be formed in the corresponding two supply flow channels 122 so as to perform two different pretreatments or two different first reactions therein. As described above, the transparent regions from the upper plate 120a and the lower plate 120b may be formed in a position corresponding to the testing chambers 125.

FIG. 12 is a plan view illustrating middle plate 120c including two inlets 121c-1 and 121c-2. As described above, the cartridge for analyzing blood 100 may include at least two supply holes 111a supplying the blood sample. When the blood supply unit 111 of the housing 110 includes at least two supply holes 111a, the testing unit 120 may also include at least two inlets 121c-1 and 121c-2 that correspond to the at least two supply holes 111a. The blood sample introduced from the two inlets 121c-1 and 121c-2 may be two different blood samples. The supply flow channels 122-1 and 122-2 each connected to the two inlets 121c-1 and 121c-2 may be connected to the plurality of the testing chambers 125-1 and 125-2, wherein the plurality of the testing chambers 125-1 and 125-2 each may be independent from the other. In some embodiments, three or more supply holes 111a may be formed, which may accordingly form three or more inlets 121c in a middle plate 120c to correspond to the three or more supply holes 111a. In this regard, the inlet 121a formed in the upper plate 120a may correspond to the inlet 121c and a supply hole 111a that are formed in the middle plate 120c. Also, the plurality of the testing chambers 125-1 and 125-2 may be arranged in a multi-layer structure. Alternatively, the plurality of the testing chambers on the top and the plurality of testing chambers on the bottom may intersect like a zigzag. In some embodiments, at least two supply channels 122 may be connected to one of the inlets 121c-1 and 121c-2.

### Example 1

### Evaluation of Filled Density of the Filter Member and Degree of Hemolysis and Recovery Rate According to Diameter of the Filter Member

A filter member made with glass fiber was punched to have a diameter in a range from about 6 to about 8.5 mm at intervals of about 0.25 mm. A supply hole includes a hole having a diameter about 0.1 mm larger than the filter member and a thickness of about 1 mm. The filter member was a MF1 filter and a VF1 filter (manufactured by the Whatman Company). A track etched membrane was disposed below the filter member, and the track etched membrane manufactured by the Whatman Company was stacked on a polycarbonate membrane having a pore size of 0.8 μm using a double-sided adhesive. That is, the MF1 filter, the VF1 filter, and the polycarbonate membrane were sequentially stacked. Such an assembled injection port was measured using a T10 device, and a testing unit on which a testing sample was not applied was used.

In order to quantify degree of hemolysis, a reagent given to hemoglobin concentration before experiment was diluted in different concentrations, and then injected into the T10 device. Accordingly, the T10 device measured a wavelength in a range from 405 to 810 nm, and a calibration curve was obtained based on the measured wavelength. Such a method may be interfered with by concentrations of other materials except hemoglobin in the blood. In this regard, 70 uL of the same whole blood was loaded in each filter so as to prevent problems that may occur due to the use of other bloods. In addition, the degree of hemolysis was measured in a way that a value obtained at the wavelength in a range from 405 to 810 nm after plasma obtained by centrifugation was injected into the T10 device was subtracted from a conversion value of hemoglobin measured by the separation of the whole blood plasma. Here, any possibility that materials in the plasma other than hemoglobin may affect changes in OD values was completely excluded.

The testing unit used in the T10 device was able to measure the recovery rate of up to 46.8%, and accordingly determine the recovery rate depending on the number of injection wells. The difference between wells before injecting the wavelength in a range from 405 to 810 nm and wells without injecting the wavelength may determine whether the injection has been occurred or not. Then, the obtained values were converted into the recovery rate.

The injection of the whole blood was performed under conditions of pressure of 8.5 kPa for 12 seconds, and the experiments were repeated three times depending on size of each filter. Accordingly, a graph was obtained as shown in FIG. 2 based on a mean value of the resulting values. The pressure conditions described above were obtained by measuring pressure changes on top of a pressure sensor that is connected with a needle from a plunger. FIG. 2 is a graph showing the degree of hymolysis and recovery rate during blood injection according to filled density and diameter of the filter member of the blood filter apparatus. As shown in FIG. 2, when the filter member had a filled density less than about 0.49 g/cm³ or greater than about 0.65 g/cm³, the recovery rate of the separated plasma or serum was as low as less than 40%. In addition, when the filter member had a filled density less than about 0.49 g/cm³, hemolysis occurred.

### Example 2

### Evaluation of Degree of Hemolysis and Recovery Rate Depending on Pressurization Time and Pressure According to Filter Member Type

Degree of hemolysis and recovery rate depending on time and pressure of pressurization were evaluated using a filter member having different particle retention sizes.

The filter member having a particle retention size of less than 1.25 may be a GA200 filter (manufactured by the Advantec Company), but is not limited thereto, and a GA100 filter (manufactured by the Advantec Company) may be also used. The filter member having a particle retention size of greater than 2.75 may be a VF2 filter (manufactured by the Whatman Company). When the VF2 filter was used, a MF1 filter or a LF1 filter (manufacture by the Whatman Company) may be used as an extra filter on top of the VF2 filter during experiments to meet thickness conditions. Alternatively, only the VF2 filter may be used during experiments. The filter member having a particle retention size in a range from about 1.25 to 2.75 may be the MF1 filter and the VF1 filter (manufactured by the Whatman company), and the LF1 filter (manufactured by the Whatman Company) may be used instead of the MF1 filter. Also, a fusion 5 filter (manufactured by the Whatman Company) may be used as a filter member having a particle retention size in a range from 1.25 to 2.75.

The filter member which was punched to have a diameter of about 7.5 mm was used, and then assembled in the same manner as in Example 1, except that there was deviation of about 0.1 mm in diameter below the supply hole.

The pressurization was performed for 6 to 18 seconds at intervals of 3 seconds. The recovery rate was indicated by obtaining a rate that reaches up to 46.8%, which is the maximum recovery rate measurable by the T10 device in 30 different whole blood samples. The degree of hemolysis was indicated in the graph based on the mean value of hemolysis obtained from the 30 different whole blood samples.

The pressure part of the blood filter apparatus may have plunger-type elasticity and use elastic materials. The pressurization was performed by conditions of the same pressure-volume for a given period of time. The pressure-volume was measured by a manometer (Handy manometer manufactured by the Copal Electronics Company) that is connected to the needle from the plunger.

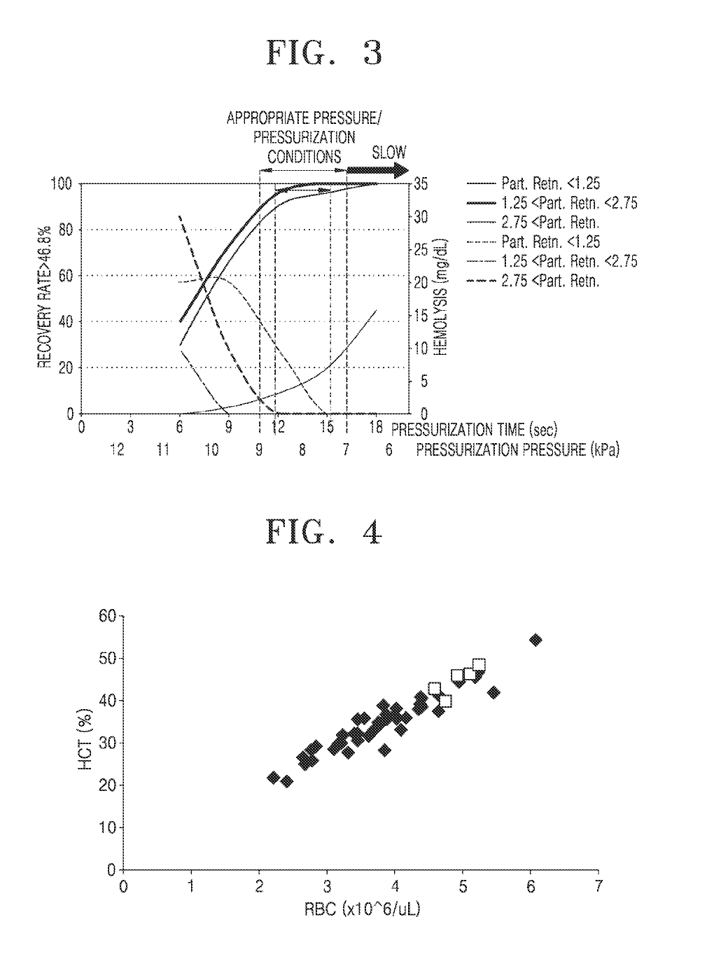

FIG. 3 is a graph showing the degree of hymolysis and recovery rate depending on pressurization time and pressure with respect to types of the filter member in the blood filter apparatus. As shown in FIG. 3, the recovery rates above 46.8% were likely to occur in the filter member having a particle retention size (Part. Retn.) less than 2.75. In addition, when the degree of hemolysis was close to 0 mg/dL, the filter member had a particle retention size of greater than 1.25 or a particle retention size of less than 2.75.

When the filter member having a particle retention size within the ranges above was pressurized for 10 to 16 seconds, the recovery rate of the separated plasma or serum was close to 100% and the degree of hemolysis thereof was close to 0 mg/dL. The pressure of the pressurization was in a range from 7 to 9 kPa. On the contrary, when the filter member was pressurized for 16 seconds or longer, the recovery rate of the separated plasma or serum was high and the degree of hemolysis thereof was small, but the separation of plasma or serum from the blood had performed for a long time.

### Example 3

### Evaluation of Hematocrit (HCT) Ranges Measurable According to Compositions of the Filter Member

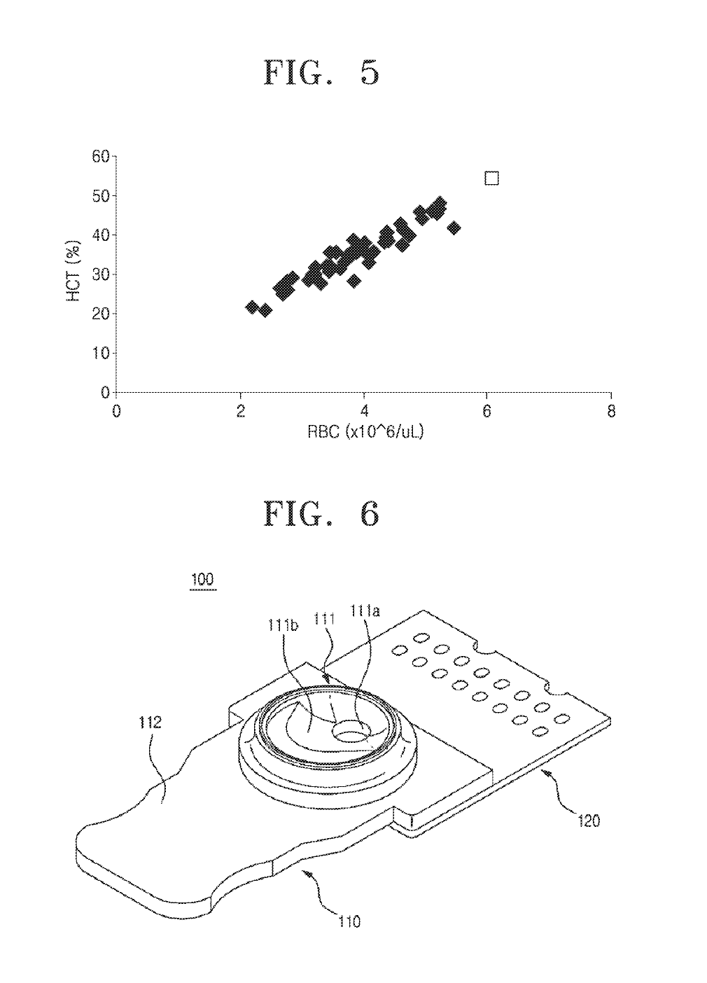

FIG. 4 is a graph showing results obtained using a GA200 filter (manufactured by the Advantech Company) with 50 different whole blood samples, and FIG. 5 is a graph showing results obtained using a VF1 filter that was disposed below an MF1 filter (manufactured by the Whatman Company) with 50 different whole blood samples, under the same conditions as in Examples 1 and 2.

The MF filter, the VF filter, and the GA200 filter were punched to have a diameter of 7.5 mm. Then, a supply hole had a fixing part for the filter member in which a diameter was about 0.1 mm larger than the filter member's and a thickness was about 1 mm was assembled with each filter member. A track etched membrane formed of polycarbonate materials having a pore size of 0.8 μm was disposed below each of the filter members using a double-sided adhesive so as to complete the assembly of the supply unit.

The pressurization was performed for 12 seconds. The pressure of the pressurization was in a range of from 8.3 to 8.7 kPa according to the blood.

Red blood cell (RBC) volume or hematocrit (HCT) in a sample was measured using a C-10 device manufactured by Samsung. Here, 70 μl of 50 different whole blood samples was supplied so as to measure experimental results. As resulted in Example 2, a graph indicated that the blood sample having the recovery rate of 46.8% or greater is normal and the blood sample having the recovery rate less than 46.8% is abnormal.

FIGS. 4 and 5 are graphs showing measurable HCT ranges with respect to compositions of the filter member in the blood filter apparatus. FIGS. 4 and 5 are graphs showing correlation between the recovery ratio and HCT and RBC. The black square represents a case where the recovery ratio is 46.8% or greater while the white square represents a case where the recovery ratio is 46.8% or less. FIG. 4 is a group showing results obtained using a GA200 filter member that has a particle retention size of 0.8. Here, when the HCT was 40% or greater than 45%, the recovery ratio was less than 46.8%. FIG. 5 is a graph showing results using a MF/VF filter member that has a particle retention size of greater than 1.25 and less than 2.75. Here, when the HCT was greater than 55%, the blood sample having the recovery ratio less than 46.8% may be present in a small amount.

As shown in FIGS. 4 and 5, it was confirmed that the filter member having a particle retention size in a range greater than 1.25 and less than 2.75 may have high recovery rate in the blood sample with a wide range of HCT.

As described above, according to the one or more of the above embodiments of the present invention, a blood filter apparatus may separate plasma or serum components from blood quickly and efficiently without causing disruption of erythrocytes. According to one or more of the above embodiments of the present invention, a cartridge for analyzing blood may accurately analyze plasma or serum components. In addition, according to one or more of the above embodiments of the present invention, a method of separating plasma or serum from a blood sample may separate plasma or serum components from blood quickly and efficiently without causing hemolysis.

It should be understood that the exemplary embodiments described therein should be considered in a descriptive sense only and not for purposes of limitation. Descriptions of features or aspects within each embodiment should typically be considered as available for other similar features or aspects in other embodiments. While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A blood filter apparatus comprising:
a plurality of filter members; and
a plasma or serum separation membrane;
wherein the plurality of filter members are serially connected to each other and disposed on the plasma or serum separation membrane, and each of the plurality of filter members have a filled density of about 0.49 to about 0.65 g/cm³.

2. The blood filter apparatus of claim 1, wherein the plurality of filter members have a basis weight in a range from about 40 to about 150 g/m², a thickness in a range from about 0.8 to about 1.2 mm, and a diameter in a range from about 7 to about 7.7 mm.

3. The blood filter apparatus of claim 1, wherein each of the plurality of filter members has a particle retention size in a range from about 1.25 to about 2.75 μm.

4. The blood filter apparatus of claim 1, wherein the plasma or serum separation membrane has a plurality of through holes penetrating from one side of the membrane to the other, wherein each through hole has a diameter in a range from about 0.2 to about 1.0 μm.

5. The blood filter apparatus of claim 1, wherein the plasma or serum separation membrane has a porosity in a range from about 10 to about 20%.

6. The blood filter apparatus of claim 1, further comprising a compressing device disposed on the plurality of filter members opposite the plasma or serum separation membrane.

7. A cartridge for analyzing blood to perform examination on a blood sample, comprising:
a testing unit configured to receive a blood sample;
a housing including at least one supply hole configured to supply the blood sample to the testing unit; and
the blood filter apparatus of claim 1 disposed in the supply hole of the housing.

8. A method of separating plasma or serum from blood, the method comprising:
providing a blood sample to the blood filter apparatus of claim 1; and
compressing the filter members of the blood filter apparatus.

9. The method according to claim 8, wherein the filter members are compressed at a pressure of about 7 to about 9 kPa.

10. The method according to claim 8, wherein the filter members are compressed for about 10 to about 16 seconds.

11. The method according to claim 8, wherein the blood sample has hematocrit (HCT) level of less than 55%.

12. The method according to claim 8, wherein the blood sample is whole blood having a volume in a range from about 70 to about 100 μl.

13. The method according to claim 8, wherein 40% or more plasma or serum is separated when the degree of hemolysis is less than 10 mg/dL.

14. The method according to claim 8, wherein the separation of the plasma or serum is performed for about 15 to about 25 seconds.

15. The method according to claim 8, further comprising washing out the blood filter apparatus with water or acid.

16. The method according to claim 15, further comprising washing out the blood filter apparatus when an amount of the residual electrolyte in the blood filter apparatus reaches a predetermined amount.
